(19) United States
(12) Patent Application Publication
(10) Pub. No.: US 2021/0145965 A1
(43) Pub. Date: May 20, 2021

(54) METHOD OF USING CHECKPOINT KINASE INHIBITOR THERAPY TO MODULATE ANTI-TUMORAL RESPONSE AGAINST CANCER AND SENSITIZE GLIOMAS TO IMMUNOTHERAPY

(71) Applicant: NORTHWESTERN UNIVERSITY, Evanston, IL (US)

(72) Inventors: Crismita C. Dmello, Chicago, IL (US); Adam M. Sonabend Worthalter, Chicago, IL (US); Catalina Lee Chang, Chicago, IL (US); Li Chen, Wilmette, IL (US)

(21) Appl. No.: 16/951,638

(22) Filed: Nov. 18, 2020

Related U.S. Application Data
(60) Provisional application No. 62/936,867, filed on Nov. 18, 2019.

(51) Int. Cl. A61K 39/395; A61K 35/17; A61K 31/4535; A61K 31/5377; A61K 31/519; A61K 31/475; A61K 31/551; A61K 31/505; A61K 31/437; A61K 31/501; A61K 31/4184; A61K 31/497; A61P 35/00

(52) U.S. Cl. CPC A61K 39/3955; A61K 35/17; A61K 31/4535; A61K 31/5377; A61K 31/519; A61K 31/475; A61K 2039/545; A61K 31/505; A61K 31/437; A61K 31/501; A61K 31/4184; A61K 31/497; A61P 35/00; A61K 31/551

(57) ABSTRACT

Disclosed are methods for treating cancer in a subject in need thereof. The methods comprise administering to the subject an inhibitor of a checkpoint kinase and administering to the subject immunotherapy.

### CROSS-REFERENCE TO RELATED APPLICATIONS

This application claims the benefit of U.S. Provisional Application No. 62/936,867, filed November 18, 2020, which is incorporated herein by reference.

### STATEMENT AS TO FEDERALLY SPONSORED RESEARCH

This invention was made with government support under 5DP5OD021356-04 awarded by NIH. The government has certain rights in the invention.

### FIELD OF INVENTION

The disclosure relates to compositions and methods for cancer treatment. In particular, the disclosure relates to compositions and methods for using checkpoint kinase inhibitor therapy to modulate anti-tumoral responses against cancers and to sensitize gliomas to immunotherapy.

### BACKGROUND

Gliomas, and in particular glioblastomas (GBM), are the most common and malignant of all primary brain tumors in adults. Unfortunately, in spite of extensive research and the use of multimodal therapeutic strategies, the median overall survival time is 20 months from diagnosis.

However, despite the use of a wide variety of immune therapy strategies, such as the use of vaccines, dendritic cells, adjuvants, adoptive cellular therapies, and immune checkpoint inhibitors, immunotherapy alone has not shown to be effective in the treatment of gliomas. In fact, immunotherapy has been shown to benefit less than 10% of GBM patients. Therefore, there exists a need to identify a method of rendering gliomas recognizable by T-cells, in order to facilitate response to immunotherapy.

### SUMMARY

In one embodiment, a method for potentiating the response of a T-cell based immunotherapy in a subject includes administration to a subject of pharmaceutically effective amount of an inhibitor of a checkpoint kinase, and an immunotherapy treatment, wherein the checkpoint kinase inhibitor potentiates the immunotherapy treatment. In one embodiment, the subject suffers from or is suspected of having a glioma, and in yet another embodiment, the glioma is a glioblastoma.

In one embodiment, the checkpoint kinase is checkpoint kinase 2, the inhibitor of checkpoint kinase 2 is prexasertib, the immunotherapy treatment comprises anti-PD-1 therapy or anti-PDL-1 therapy, and the immunotherapy treatment is directed to potentiation of an ERK pathway.

It should be understood that the checkpoint kinase inhibitor and the immunotherapy treatment may be administered on different dosing schemes. In one embodiment, the method may include providing instructions for determining the dosing scheme for administration to a subject of the effective amount of a checkpoint kinase inhibitor and effective amount of an immunotherapy treatment.

### BRIEF DESCRIPTIONS OF DRAWINGS

FIG. 1 is schematic representation of an in vivo method useful for identifying the protein kinases that are effective as resistance conferring genes against T-cell mediated killing.

FIG. 2 is a graphical representation comparing the percentage of CD45+ lymphocytes present in different immune populations from the spleen from CD8wild type mice and CD8 T-cell KO mice, using flow cytometry.

FIG. 3 is a Kaplan-Meier survival curve comparing the survival of untreated wild type mice with CD8 T-cell KO mice that have been implanted with kinome KO GL261 glioma cells.

FIG. 4 is a scatter plot of data obtained using CRISPR screening technology, showing the genes most enriched in the CD8 T-cell KO mice, as compared to the untreated wild type mice.

FIG. 5 is a graphical representation of the fold change for the two most enriched Checkpoint kinase 2 (CHEK2) sgRNA in the CD8 T-cell KO mice, as compared to 100 non-targeting sgRNA for the wild type mice.

FIG. 6 is a graphical representation using the GlioVis TCGA RNA data portal comparing the expression of CHEK2 in GBM patients and the non-tumor controls.

FIG. 7 is a graphical representation comparing the percent viability of GL261 cells treated with varying amounts of 1) temozolomide (TMZ) and 2) iCHEK1/2.

FIG. 8 is a western blot image comparing the amount of DNA damage to GL261 cells treated with 8 nM of iCHEK1/2 (IC50 concentration of CHEK2) and 50 μM of TMZ, respectively, and GL261 cells that were untreated over a 48-hour period.

FIG. 9 is a graphical representation comparing the expression of MHC-I and PDL1 in GL261 cells that were 1) untreated and 2) treated with 0.9 nM iCHEK 1/2 (CHEK1 IC50) and 8 nM iCHEK 1/2 (CHEK2 IC50), respectively, over two cycles (48 hours).

FIG. 10 is a graphical representation comparing the expression of MHC-I in GL261 cells that were 1) untreated, 2) treated with TMZ, and 3) treated with iCHEK1/2 (IC 50=8 nM), respectively, over the course of three cycles (48 hour).

FIG. 11 is a graphical representation comparing the percentages of PD1+ T cells present in the brain and spleen of iCHEK1/2 treated mice, untreated mice, and mice treated with TMZ.

FIG. 12 is a western blot image confirming the knockout of CHEK2 in the GL261 cells.

FIG. 13 is a Kaplan-Meier survival curve for mice injected with control wild type GL261 cells and treated with anti-PD1 monoclonal antibodies or isotype antibodies.

FIG. 14 is a Kaplan-Meier survival curve for mice injected with CHEK2 KO GL261 cells and then treated with anti-PD1 monoclonal antibodies or Isotype antibodies.

FIG. 15 is a Kaplan-Meier survival curve showing the survival of GL261-bearing mice that received injections of 1) isotype antibodies, 2) anti-PD1 monoclonal antibodies, 3) iCHEK1/2 (prexasertib), and 4) a combination of anti-PD1 monoclonal antibodies and iCHEK1/2 (prexasertib).

FIG. 16 is a Kaplan-Meier survival curve showing the survival of surviving mice re-challenged with GL261 cells on a brain hemisphere, opposite of the initial tumor injection site, with 1) isotype antibodies, 2) anti-PD1 monoclonal antibodies, and 3) anti-PD1 monoclonal antibodies and iCHEK 1/2; 172 days after initial exposure.

FIG. 17 is a graphical representation comparing of the presence of CD8 T-cells in re-challenged long-term survivors (LTS-1 and LTS-2), non-tumor bearing mice, and tumor bearing control mice. The samples were further analyzed for the presence of lymphocyte phenotype. And, the LTS-1, LTS-2, and tumor bearing control mice were analyzed for their ability to express intracellular GzMB, TNFα, and IFNγ.

### DETAILED DESCRIPTION

A method for treating cancer in a subject in need thereof is disclosed. In one embodiment, the method includes administering to the subject an inhibitor of a checkpoint kinase and administering to the subject immunotherapy. In one embodiment, the cancer is glioma, and more specifically, glioblastoma (GBM), the checkpoint kinase is CHEK2, the inhibitor of the checkpoint kinase is prexasertib, and the immunotherapy comprises administering to the subject anti-PD-1 (programmed cell death protein 1) therapy or anti-PDL-1(programmed cell death-ligand 1) therapy.

It should be understood that while Prexasertib (LY2606368) has been used an example of a suitable CHEK2 inhibitor (or iCHEK1/2), any checkpoint kinase inhibitor effective for inhibiting the expression of CHEK2 is contemplated. For the purposes of this disclosure, iCHEK1/2 means an inhibitor of checkpoint kinase 1 and/or 2. For example, suitable checkpoint kinase inhibitors of CHECK1 and CHECK2 include, but are not limited to, Prexasertib, AZD7762, Rabusertib (LY2603618), MK-8776 (SCH 900776), CHIR-124, PF-477736, VX-803 (M4344), DB07268, GDC-0575 (ARRY-575), SAR-020106, CCT245737, PD0166285, Chk2 Inhibitor II (BML-277), Prexasertib HCl (LY2606368), and combinations thereof.

The iCHEK1/2 composition may be administered in a dosages suitable to potentiate the desired immunotherapy treatment. In one embodiment, Prexasertib may be administered in a dose of about 105 mg/m² IV once every 14 days for a 28-day cycle.

Moreover, any suitable immunotherapy treatment may be used in combination with the checkpoint kinase inhibitor. Suitable immunotherapy treatments include, but are not limited to the administration of anti-PD-1 or anti-PD-L1 monoclonal antibodies (PD-1 and PD-L1 inhibitors). For example, IgG4 PD1 antibodies (Nivolumab, Pembrolizumab, Cemiplimab) and BGB-A317 are known PD-1 inhibitors suitable for use in combination with the iCHEK1/2 composition. In addition, PD-L1 inhibitors, such as atezolizumab, avelumab, and durvalumab and checkpoint inhibitor drugs that target CTLA-4, such as Ipilimumab (commercially available as Yervoy), may also be useful immunotherapies. T-cell therapies, such CAR T and BiTE, may be potentiated by the use of the iCHEK1/2 composition. Specific immunotherapy compositions may be administered as single composition dosages or may be administered in combination with other immunotherapy compositions.

In one embodiment, Nivolumab may be administered in combination with the iCHEK1/2 composition. Specifically, each patient may receive two doses of nivolumab prior to radiation. Nivolumab may be administered at a dose of 3 mg/kg, given intravenously, before re-RT (day 1+/−5 and 14+/−5) and when given with bevacizumab (day 28+/−5 (medical arm only), day 42+/−5, and day 56+/−5). The dosage for Nivolumab, for example, maybe dosed based on body weight, while combined with re-radiation and bevacizumab for safety considerations to reduce adverse events. In another embodiment, single agent nivolumab may be given at 240 mg flat dose every 2 weeks thereafter until disease progression, withdrawal, adverse event, or death.

Among all the genes in the human genome, kinases are potentially targetable modulators of the body's response to immunotherapy. Furthermore, GBM patient cohorts have shown enrichment of BRAF/PTPN11 mutations (members of the MAP kinases) among tumors responsive to anti-PD-1 therapy (Zhao et al., Nature Med 2019). Therefore, in vivo kinome knockout CRISPR screens were performed to identify kinases that most likely impair CD8 T-cells ability to recognize and target glioma cells. It was determined that CHEK2, which has a central role in DNA damage response, was the most effective among kinases in rendering GBMs resistant to CD8 T-cell recognition. Therefore, it was determined that pharmacological targeting of CHEK2 enhances glioma susceptibility to T-cell recognition and facilitates the response of these tumors to immunotherapy.

Applications of the disclosed subject matter include, but are not limited to: (i) Pharmacologic CHEK inhibition as a therapy for gliomas that will promote recognition of tumor cells by anti-tumoral immunity and enhanced response to immunotherapy; (ii) CHEK inhibition can be potentially combined with different T-cell therapies like immune Checkpoint blockade and CAR T cell therapy; and (iii) this combination therapy can be used to treat melanoma, lung and breast cancers that are found to refractory to immunotherapy.

### Definitions

As used herein, the term “tumor” refers to any neoplastic growth, proliferation or cell mass whether benign or malignant (cancerous), whether a primary site lesion or metastases.

As used herein “therapeutically effective amount” refers to an amount of a composition that relieves (to some extent, as judged by a skilled medical practitioner) one or more symptoms of the disease or condition in a mammal. Additionally, by “therapeutically effective amount” of a composition is meant an amount that returns to normal, either partially or completely, physiological or biochemical parameters associated with or causative of a disease or condition. A clinician skilled in the art can determine the therapeutically effective amount of a composition in order to treat or prevent a particular disease condition, or disorder when it is administered, such as intravenously, subcutaneously, intraperitoneally, orally, or through inhalation. The precise amount of the composition required to be therapeutically effective will depend upon numerous factors, e.g., such as the specific activity of the active agent, the delivery device employed, physical characteristics of the agent, purpose for the administration, in addition to many patient specific considerations. But a determination of a therapeutically effective amount is within the skill of an ordinarily skilled clinician upon the appreciation of the disclosure set forth herein.

Treat”, “treating”, and “treatment”, etc., as used herein, refer to any action providing a benefit to a patient at risk for or afflicted with a disease, including improvement in the condition through lessening or suppression of at least one symptom, delay in progression of the disease, prevention or delay in the onset of the disease, etc. Treatment also includes partial or total destruction of the undesirable proliferating cells with minimal destructive effects on normal cells. A subject at risk is a subject who has been determined to have an above-average risk that a subject will develop cancer, which can be determined, for example, through family history or the detection of genes causing a predisposition to developing cancer.

The term “subject,” as used herein, refers to a species of mammal, including, but not limited to, primates, including simians and humans, equines (e.g., horses), canines (e.g., dogs), felines, various domesticated livestock (e.g., ungulates, such as swine, pigs, goats, sheep, and the like), as well as domesticated pets and animals maintained in zoos.

Pharmaceutical compositions may include carriers, thickeners, diluents, buffers, preservatives, surface active agents and the like in addition to the molecule, in this case virus or viral vector, of choice. Pharmaceutical carriers are known to those skilled in the art. These most typically would be standard carriers for administration of drugs to humans, including solutions such as sterile water, saline, and buffered solutions at physiological pH. Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of a pharmaceutically-acceptable carriers include, but are not limited to, saline, Ringer's solution and dextrose solution. The pH of the solution is preferably from about 5 to about 8, and more preferably from about 7 to about 7.5. Further carriers may include sustained release preparations such as semipermeable matrices of solid hydrophobic polymers containing the antibody, which matrices are in the form of shaped articles, e.g., films, liposomes or microparticles. It will be apparent to those skilled in the art that certain carriers may be more preferable depending upon, for instance, the route of administration and concentration of composition being administered.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as; for example, antimicrobials, anti-oxidants, chelating agents, and inert gases and the like.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

Effective dosages and schedules for administering the compositions may be determined empirically, and making such determinations is within the skill in the art. For example, there are several brain tumor models that provide a mechanism for rapid screening and evaluation of potential toxicities and efficacies of experimental therapies. There are six separate human glioma xenograft models used for critical studies. Pandita et al., Genes Chromosomes Cancer 39(1): 29-36 (2004). There is also available a spontaneously derived syngeneic glioma model that does not express foreign antigens commonly associated with chemically or virally induced experimental tumors. Hellums et al., Neuro-oncol. 7(3): 213-24 (2005). Other animal models for a variety of cancers can be obtained, for example, from The Jackson Laboratory, 600 Main Street Bar Harbor, Me. 04609 USA, which provides hundreds of cancer mouse models. Both direct (histology) and functional measurements (survival) of tumor volume can be used to monitor response to oncolytic therapy. These methods involve the sacrifice of representative animals to evaluate the population, increasing the animal numbers necessary for the experiments. Measurement of luciferase activity in the tumor provides an alternative method to evaluate tumor volume without animal sacrifice and allowing longitudinal population-based analysis of therapy.

The dosage ranges for the administration of the compositions are those large enough to produce the desired effect in which the symptoms of the disease are affected. The dosage should not be so large as to cause adverse side effects, such as unwanted cross-reactions, anaphylactic reactions, and the like. The dosage can be adjusted by the individual physician in the event of any counterindications. Dosage can vary, and can be administered in one or more dose administrations daily, for one or several days.

In general, any combination therapy will include one or more of chemotherapeutics, targeting agents like antibodies; kinase inhibitors; hormonal agents and the like. Combination therapies can also include conventional therapy, including, but not limited to, antibody administration, vaccine administration, administration of cytotoxic agents, natural amino acid polypeptides, nucleic acids, nucleotide analogues, and biologic response modifiers. Two or more combined compounds may be used together or sequentially. As used herein, a first line “chemotherapeutic agent” or first line chemotherapy is a medicament that may be used to treat cancer, and generally has the ability to kill cancerous cells directly.

Examples of chemotherapeutic agents include alkylating agents, antimetabolites, natural products, hormones and antagonists, and miscellaneous agents. Examples of alkylating agents include nitrogen mustards such as mechlorethamine, cyclophosphamide, ifosfamide, melphalan (L-sarcolysin) and chlorambucil; ethylenimines and methylmelamines such as hexamethylmelamine and thiotepa; alkyl sulfonates such as busulfan; nitrosoureas such as carmustine (BCNU), semustine (methyl-CCNU), lomustine (CCNU) and streptozocin (streptozotocin); DNA synthesis antagonists such as estramustine phosphate; and triazines such as dacarbazine (DTIC, dimethyl-triazenoimidazolecarboxamide) and temozolomide. Examples of antimetabolites include folic acid analogs such as methotrexate (amethopterin); pyrimidine analogs such as fluorouracin (5-fluorouracil, 5-FU, 5FU), floxuridine (fluorodeoxyuridine, FUdR), cytarabine (cytosine arabinoside) and gemcitabine; purine analogs such as mercaptopurine (6-niercaptopurine, 6-MP), thioguanine (6-thioguanine, TG) and pentostatin (2′-deoxycoformycin, deoxycoformycin), cladribine and fludarabine; and topoisomerase inhibitors such as amsacrine. Examples of natural products include vinca alkaloids such as vinblastine (VLB) and vincristine; taxanes such as paclitaxel (Abraxane) and docetaxel (Taxotere); epipodophyllotoxins such as etoposide and teniposide; camptothecins such as topotecan and irinotecan; antibiotics such as dactinomycin (actinomycin D), daunorubicin (daunomycin, rubidomycin), doxorubicin, bleomycin, mitomycin (mitomycin C), idarubicin, epirubicin; enzymes such as L-asparaginase; and biological response modifiers such as interferon alpha and interlelukin 2. Examples of hormones and antagonists include luteinizing releasing hormone agonists such as buserelin; adrenocorticosteroids such as prednisone and related preparations; progestins such as hydroxyprogesterone caproate, medroxyprogesterone acetate and megestrol acetate; estrogens such as diethylstilbestrol and ethinyl estradiol and related preparations; estrogen antagonists such as tamoxifen and anastrozole; androgens such as testosterone propionate and fluoxymesterone and related preparations; androgen antagonists such as flutamide and bicalutamide; and gonadotropin-releasing hormone analogs such as leuprolide. Examples of miscellaneous agents include thalidomide; platinum coordination complexes such as cisplatin (czs-DDP), oxaliplatin and carboplatin; anthracenediones such as mitoxantrone; substituted ureas such as hydroxyurea; methylhydrazine derivatives such as procarbazine (N-methylhydrazine, MIH); adrenocortical suppressants such as mitotane (o,p′-DDD) and aminoglutethimide; RXR agonists such as bexarotene; and tyrosine kinase inhibitors such as imatinib.

Potentiation: Potentiation describes a generally synergistic effect when, for example, a checkpoint kinase inhibitor therapeutic regimen is added to another immunotherapy treatment regimen for more effective treatment of a subject.

### EXAMPLES

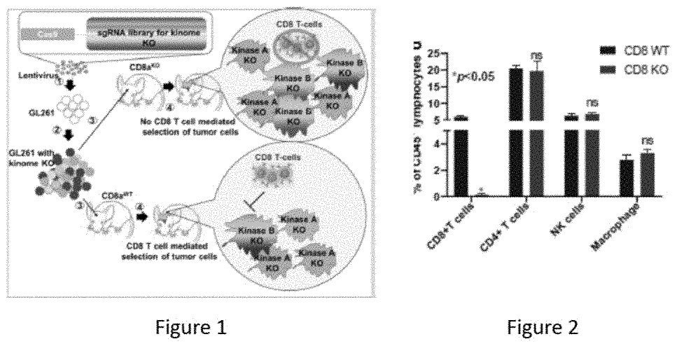

As shown in FIG. 1, an in vivo CRISPR screening was used to identify kinases that impair glioma cell recognition by CD8 T-cells. In this example an in vivo kinome knockout CRISPR screen was performed using intact immunity (or wild type) mice and CD8 T-cell knockout mice to determine the kinase most likely to affect the body's CD8 T-cell ability to recognize and target glioma cells. In this experiment, mouse glioma 261 (G1261) cells were knocked out for every kinase in the genome and were implanted intracranially in C57B/L6 strain, with either wild type immunity or with CD8 T-cell knockout (KO) background. Kinases selected by T-cells were identified by comparing the individual kinase guide counts in wild type (CD8 WT) versus that in CD8 T KO (CD8 KO) mice. As shown in FIG. 2, the percentages of different immune populations in the spleen from wild type and CD8 T-cell KO mice, using flow cytometry, were measured. It was confirmed that the percentage of CD45 lymphocytes for the CD8 T-cell population was removed from the CD8 KO mice.

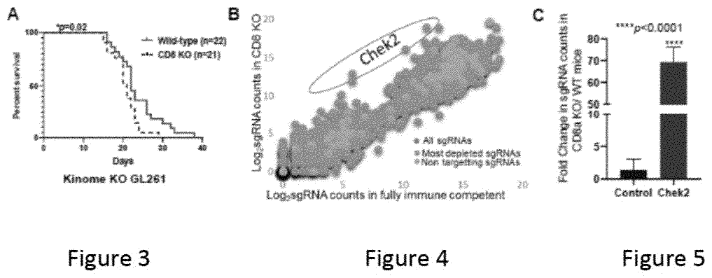

In addition, the in vivo kinome KO CRISPR screen revealed that Checkpoint kinase 2 (CHEK2) was the most depleted kinase in the CD8 KO mice, and therefore CHEK2 favored glioma escape from T-cell recognition. As shown in FIG. 3, a Kaplan-Meier curve evidenced a significant difference in survival in CD8 WT as compared to the CD8 KO mice, injected with kinome KO GL261. Log-rank (Mantel-Cox) test was used to determine statistical significance.

As shown in FIG. 4, a scatter plot depicting the kinase with highest sgRNA depletion in the CD8 WT, as compared to the CD8 KO mice, was performed. It was determined that CHEK2 showed the most depletion of sgRNAs. This was confirmed by comparing fold change for frequency of KO glioma clones in CD8 KO mice over CD8 WT mice for the two most enriched sgRNA was compared to 100 non-targeting control sgRNA, as shown in FIG. 5.

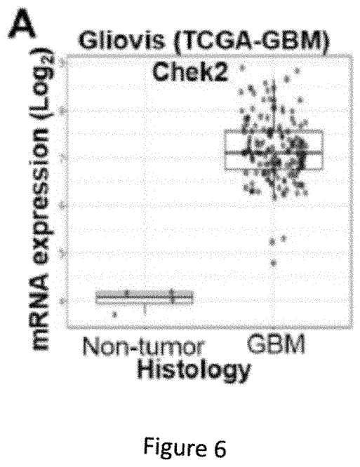

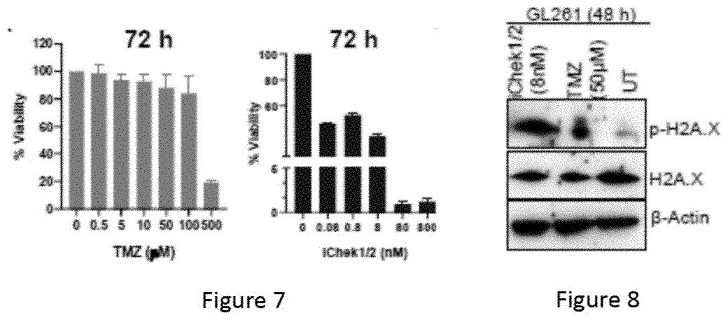

To further confirm that CHEK2 was the most likely kinase to affect CD8 T-cells ability to recognize glioma cells, a GlioVis TCGA RNA seq data plot was performed, as shown in FIG. 6. It was determined that a high expression of CHEK2 in GBM patients was present, as compared to non-tumor controls. Further, as shown in FIG. 7, treatment with TMZ for 72 h was shown to lead to a decrease in viability of glioma cells at 500 μM, while iCHEK1/2 treatment, in one embodiment Prexasertib (a CHEK inhibitor for CHEK1 and CHEK2), started to affect viability of the GL261 cells at less than 0.08 nM concentration.

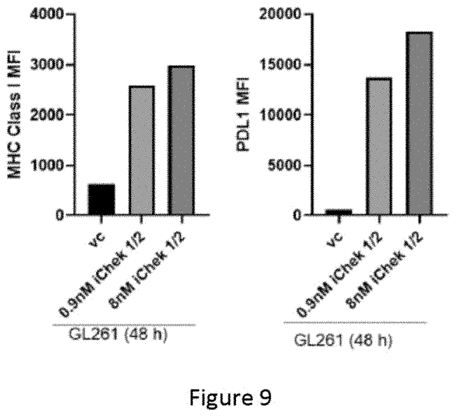

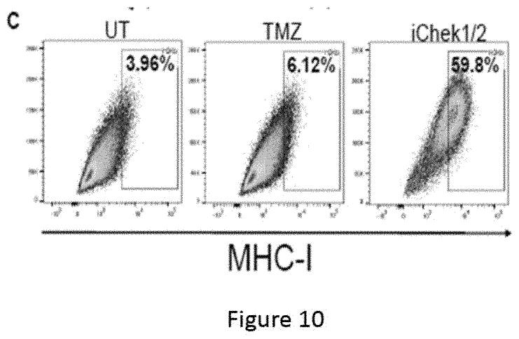

As shown in FIG. 8, the administration of 8 nM of iCHEK1/2 (IC50 concentration of CHEK2) showed an intense signal of DNA damage, as compared to TMZ (the standard of care drug) or untreated (UT) in GL261 cells upon 48 h of treatment. And, as shown in FIGS. 9 and 10, in-vitro treatment with iCHEK1/2 (IC50=8 nM) in GL261 cells for 3 cycles (48 h/cycle), resulted in increased expression of MHC-I (both MHC class and PDL1), as compared to similar treatment regimen of TMZ or in untreated (UT) subjects. Therefore, it was determined that the depletion of CHEK2 resulted in increased DNA damage and MHC-I expression.

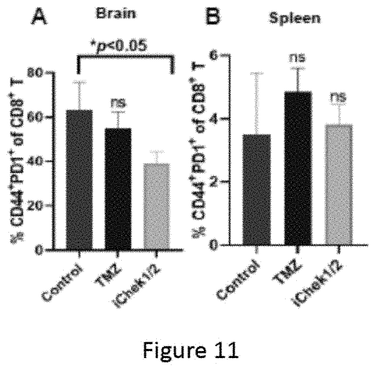

It was also determined that systemic CHEK2 inhibition decreases exhaustion of CD8 T-cell in the brains of glioma bearing mice. The systemic delivery of a CHEK2 inhibitor increased activated CD8 T-cell pools in the brains of mice bearing GL261 intracranial gliomas. Specifically, iCHEK1/2 (10 mg/kg) i.p., TMZ (10 mg/kg) i.p., and vehicle (DMSO) doses were administered on the 7th and 9th day to the GL261 intracranial tumor bearing C57BL/6 mice. The animals were sacrificed on the 13th day and the brains and spleens were harvested to check the effect on activated CD8 T-cell population by flow cytometry. As shown in FIG. 11, a decrease in the percentage of PD1+ T cells in iCHEK1/2 treated mice was detected, as compared to the TMZ or the vehicle treated control mice (n=3/group). It should be noted that no significant difference was seen in the activated T-cells pool in the peripheral compartment (spleen).

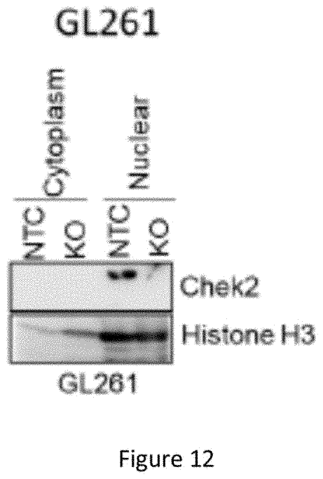

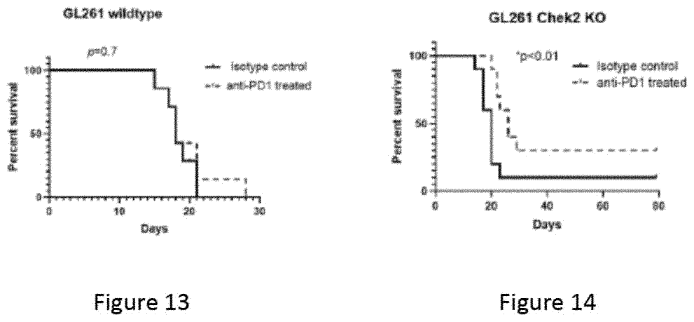

Referring now to FIGS. 12, 13, and 14, the knockout of CHEK2 in GL261 glioma cells makes the tumor more responsive to PD1 immunotherapy. Specifically, FIG. 12 confirms the knockout of CHEK2 in GL261. In one example, mice having intracranial tumors were treated with either PD1 or isotype antibodies on the 7th, 10th, 14th, and 17th days after they were injected with GL261 glioma cells (wild type and CHEK2 KO). As shown in FIGS. 13 and 14, the Kaplan-Meier survival curves for the mice injected with wild type GL261 and CHEK2 KO G1261 were compared. It was found that the mice injected with the CHEK2 KO GL261 cells survived significantly longer when treated with PD1 antibodies, compared to those that were not. Specifically, for wild type G1261 mice, the median survival was 18 days for both isotype antibody and PD1 antibody treated groups (p<0.7, number of mice; n=10/group). For the CHEK2 KO G1261 mice, the median survival was 20 days for isotype antibodies versus 26 days for the PD1 antibody treated group (p<0.01; n=10/group).

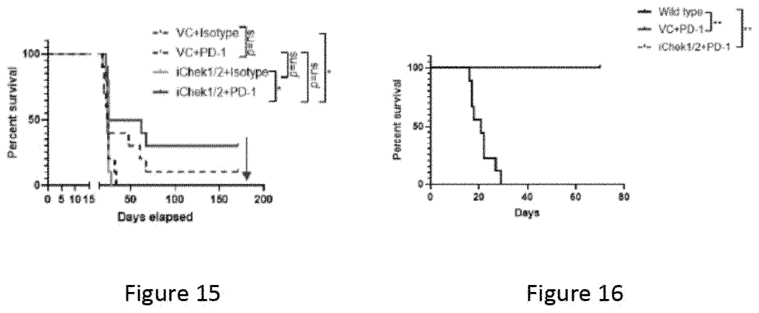

Therefore, this data indicates that drugs inhibiting CHEK2 and activating ERK (extracellular signal-related kinase) pathways can be used to potentiate response to T-cell based immunotherapies in gliomas. Moreover, once it was determined that the CHEK2 KO GL261 mice treated with PD1 antibodies had an increased survival rate, it could be surmised that the combination treatment of a CHEK2 inhibitor with immunotherapy should provide synergistic results to treat gliomas. Referring now to FIGS. 15, 16, and 17, it was determined that the combination of pharmacological inhibition of CHEK1/2 and PD1 immunotherapy in GL261 glioma bearing mice lead to the eradication of tumors in 30% of mice. For this experiment, GL261 bearing mice received isotype antibodies (control) (Group 1), injections of anti-PD1 (Group 2), iCHEK1/2 (prexasertib) (Group 3), or a combination of anti-PD1 and iCHEK1/2 (Group 4). The dosing scheme was as follows: Days 7, 8, 9, 13, 14, 15, 19, 20, 21=iCHEK1/2; Days 10, 12, 16, 18, and 22=anti-PD1. The combination therapy mice received iCHEK1/2on Days 7, 8, 9, 13, 14, 15, 19, 20, 21 and also received anti-PD1 on Days 10, 12, 16, 18, and 22. The control animals received vehicle for iCHEK1/2 which is 20% captisol in water on Days 7, 8, 9, 13, 14, 15, 19, 20, 21 and Isotype antibody on Days 10, 12, 16, 18, and 22. After 172 days after tumor challenge, the surviving mice from Groups 3 and 4 were re-challenged with GL261 cells on another hemisphere, opposite of the initial tumor injection site. The initial challenge to the mice resulted in the Kaplan-Meier survival curve shown in FIG. 15. It was determined that the mice given the combination therapy had a higher survival rate that the other three groups. Moreover, of the surviving mice from groups 3 and 4, when re-challenged, 100% of those treated with either anti-PD1 or a combination of anti-PD1 and iCHEK 1/2 survived, while by Day 30 all of the wild type mice had perished, as shown in FIG. 16.

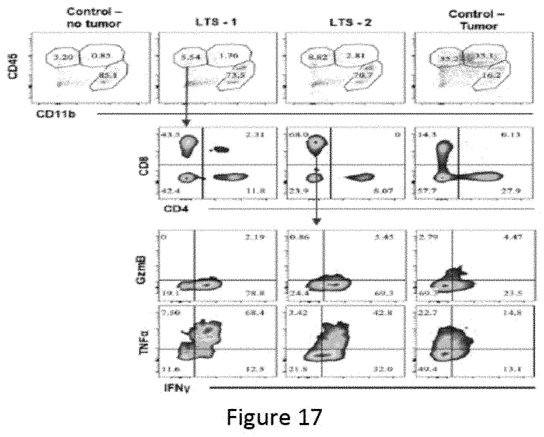

Finally, the re-challenged long term survivors (LTS) were sacrificed and checked for the presence of CD8 T-cells, as shown in FIG. 17. Non-tumor bearing (no tumor) and age-matched GL261-bearing mice (Control) were used as control groups. The freshly dissected brains from the no tumor and control groups were compared with two batches of LTS mice, LTS-1 and LTS-2 (those treated with the combination of iCHEK1/2 and anti-PD1 monoclonal antibodies) to analyze the amount of lymphocytes phenotype. Thereafter, CD8 T-cell subsets from the Control group and LTS-1 and LTS-2 groups were further tested for their ability to express intracellular GzmB, TNFα, and IFNγ.

It will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

Citations to a number of patent and non-patent references may be made herein. The cited references are incorporated by reference herein in their entireties. In the event that there is an inconsistency between a definition of a term in the specification as compared to a definition of the term in a cited reference, the term should be interpreted based on the definition in the specification.

We claim:

1. A method for potentiating the response of a T-cell based immunotherapy in a subject, the method comprising:
administration to a subject of pharmaceutically effective amount of:
an inhibitor of a checkpoint kinase, and
an immunotherapy treatment,
wherein the checkpoint kinase inhibitor potentiates the immunotherapy treatment.

2. The method of claim 1, wherein the subject suffers from or is suspected of having a glioma.

3. The method of claim 2, wherein the glioma is a glioblastoma.

4. The method of claim 1, wherein the checkpoint kinase is checkpoint kinase 2 or checkpoint kinase 1.

5. The method of claim 4, wherein the inhibitor comprises AZD7762, rabusertib, MK-8776, CHIR-124, PF-477736, VX-803, DB07268, GDC-0575, SAR-020106, CCT245737, PD0166285, Chk2 Inhibitor II (BML-277), Prexasertib HCl, and combinations thereof.

6. The method of claim 5, wherein the inhibitor comprises Prexasertib HCl.

7. The method of claim 1, wherein the immunotherapy treatment comprises anti-PD-1 therapy or anti-PDL-1 therapy.

8. The method of claim 7, wherein the immunotherapy treatment comprises anti-PD-1 therapy, and wherein the anti-PD-1 therapy comprises the administration of therapeutically effective amounts of an IgG4 antibody composition.

9. The method of claim 8, wherein the IgG4 antibody composition comprises therapeutically effective amounts of nivolumab, pembrolizumab, cemiplimab, BGB-A317, and combinations thereof.

10. The method of claim 7, wherein the immunotherapy treatment comprises anti-PD-L1 therapy, and wherein the anti-PD-L1 therapy comprises the administration of therapeutically effective amounts of atezolizumab, avelumab, durvalumab, and combinations thereof.

11. The method of claim 7, wherein the immunotherapy treatment comprises the administration of therapeutically effective amounts of a checkpoint inhibitor that targets cytotoxic T-lymphocyte-associated protein 4.

12. The method of claim 11, wherein the immunotherapy treatment comprises a therapeutically effective amount of ipilimumab.

13. The method of claim 7, wherein the immunotherapy treatment comprises T-cell therapies.

14. The method of claim 1 wherein the immunotherapy is directed to potentiation of an ERK pathway.

15. The method of claim 1 further comprising administration to the subject a pharmaceutically effective amount of a chemotherapeutic treatment.

16. The method of claim 1, wherein the pharmaceutically effective amount of a checkpoint kinase inhibitor comprises about 8 nM of checkpoint kinase inhibitor.

17. The method of claim 1, wherein the checkpoint kinase inhibitor and the immunotherapy treatment are on different dosing schemes.

18. A composition comprising an effective amount of a checkpoint kinase 2 inhibitor, and an effective amount of an immunotherapy treatment.

19. The composition of claim 18, further comprising instructions for determining a dosing scheme for administration to a subject of the effective amount of a checkpoint kinase inhibitor and effective amount of an immunotherapy treatment.
